(19) United States
(12) Patent Application Publication
(10) Pub. No.: US 2004/0096515 A1
(43) Pub. Date: May 20, 2004

(54) Methods and compositions for encapsulating active agents

(76) Inventors: Andreas R. Bausch, Munchen (DE); Anthony D. Dinsmore, Amherst, MA (US); Ming F. Hsu, Somerville, MA (US); Michael Nikolaides, Munchen (DE); David A. Weitz, Bolton, MA (US)

Correspondence Address: HALE AND DORR, LLP, 60 STATE STREET, BOSTON, MA, 02109

(21) Appl. No.: 10/433,753

(22) PCT Filed: Dec. 7, 2001
(86) PCT No.: PCT/US01/46181

(51) Int. Cl. A61K 9/16; B01J 13/02; A61K 9/50

(52) U.S. Cl. 424/490; 264/4.1

(57) ABSTRACT

Methods for making self-assembled, selectively permeable elastic microscopie structures, referred to herein as colloidosomes, that have controlled pore-size, porosity and advantageous mechanical properties are described. In one form of the invention, a method of forming colloidosomes includes providing particles formed from a biocompatible material in a first solvent and forming an emulsion by adding a first fluid to the first solvent wherein the emulsion is defined by droplets of the first fluid surrounded by the first solvent. The method includes coating the surface of droplet with the particles and the stabilizing the particles on the surface of droplet. The colloidosomes produced typically have a yield strength of at least about 20 Pascals. In certain forms of the invention, the particles are spherical and are formed of a biocompatible polymer. Colloidosomes formed according to the methods described herein are also provided. In one form, a colloidosome includes a shell formed of biocompatible, substantially spherical particles wherein each of the particles are linked to neighboring particles. The shell defines an inner chamber sized for housing a desired active agent and has a plurality of pores extending therethrough. The colloidosomes are structurally stable, typically having a yield strength of at least about 20 Pascals. Colloidal suspension and methods of encapsulating a desired active agent are also described.

### CROSS-REFERENCE TO RELATED APPLICATIONS

The present application claims the benefit of U.S. provisional patent application Ser. No. 60/254,210, filed on Dec. 7, 2000, which is hereby incorporated by reference in its entirety.

The present invention was made with Government support under grant number DRM-9971432 awarded by the National Science Foundation, and NAG3-2284 awarded by the National Aeronautics and Space Administration. The Government has certain rights in the invention.

### BACKGROUND OF THE INVENTION

The present invention relates generally to methods for making self-assembled, selectively permeable elastic microscopic structures that have controlled poresize, porosity and superior mechanical properties, as well as the structures formed and various uses thereof.

Many technologies require flexible methods to prepare new materials with architecture that is controlled at the length scale of nanometers and microns. For example, encapsulation and controlled release of foods, drugs or living cells require precise control of a capsule's size and permeability to cells, proteins or other biological macromolecules. Additional requirements include the ability to fabricate capsules from a wide variety of inorganic or organic materials, to control their mechanical strength, and to fill the capsules efficiently and without exposing the encapsulated material to damaging environments. Although a variety of techniques have been developed to address specific needs, a universal and flexible approach has been lacking.

In determining an approach to fabricate nano-or micro-porous capsules, encapsulation of living cells in alginate has been accomplished. Additionally, electrostatic deposition of alternating layers of particles on the surfaces of living cells provides a flexible approach. However, these approaches are not readily generalized for encapsulation of other materials. Additionally, the alginate capsules may not provide a sufficiently narrow distribution of pore sizes to prevent isolation of the encapsulated cell from various immune system components, such as antibodies. Other approaches, such as use of microfabrication technology, require demanding lithographic capabilities, yield only one capsule at a time and are not easily applicable to polymeric or other inorganic molecules. Thus, alternative, general approaches for preparation of elastic, micron-to-millimeter sized capsules that exhibit size-selective permeability are needed. The present invention addresses this need.

### SUMMARY OF THE INVENTION

Methods for making self-assembled, selectively permeable elastic microscopic structures, referred to herein as colloidosomes, that have controlled pore-size, porosity and desired mechanical properties have been discovered. Accordingly, methods of forming colloidosomes are provided.

In one aspect of the invention, a method of forming colloidosomes includes providing particles formed from a biocompatible material in a first solvent and forming an emulsion by adding a first fluid to the first solvent wherein the emulsion is defined by droplets of the first fluid surrounded by the first solvent. The method includes coating the surface of the droplets with the particles and then stabilizing the particles on the surface of the droplets to form stable colloidosomes. The colloidosomes produced typically have a yield strength of at least about 20 Pascals. The method may be performed with an oil-in-water system or a water-in-oil system. In at least some embodiments of the invention, the particles are spherical and are formed from a biocompatible polymer.

In yet another aspect of the invention, methods of encapsulating an active agent are provided. In one embodiment, a method includes providing particles formed from a biocompatible material in a first solvent and forming an emulsion by adding a second solvent containing the active agent to the first solvent. The emulsion is defined by droplets of the second solvent surrounded by the first solvent. The method includes coating the surface of the droplets with the particles and stabilizing the particles on the surface of the droplets to form stable colloidosomes. The colloidosomes typically have a yield strength of at least about 20 Pascals. In at least some other embodiments of the invention, the particles are substantially spherical and are formed from a biocompatible polymer.

In a further aspect of the invention, colloidosomes formed from the methods described herein are provided. In at least some embodiments, a colloidosome includes a shell formed of biocompatible, substantially spherical particles wherein each of the particles are linked to neighboring particles. The shell defines an inner chamber and has a plurality of pores extending therethrough. The chamber in certain embodiments is sized for housing an active agent. The colloidosomes typically have a yield strength of at least about 20 Pascals. The particles that form the colloidosome may be linked by a variety of methods to stabilize the colloidosome, including use of van der Waals forces, polyelectrolytes, by a swelling method or by a sintering process.

In other aspects of the invention, a colloidosome suspension is provided. In at least some embodiments, the suspension includes a colloidosome suspended in a first solvent wherein the colloidosome has a shell formed of biocompatible, substantially spherical particles. Each of the particles are linked to neighboring particles. The shell defines an inner chamber and has a plurality of pores extending therethrough. The chamber is sized for housing an active agent and filled with a second solvent that is substantially identical to the first solvent.

### BRIEF DESCRIPTION OF THE FIGURES

FIG. 1 depicts a drawing of the steps in a method of forming a colloidosome in a water-in-oil system described herein. fexample 5

FIG. 2 depicts a drawing showing a cross-sectional view of a colloidosome in decalin formed according to the methods described herein. PMMA, polymethylmethacrylate.

FIG. 3 depicts a side view of self-assembled particles forming the shell of a colloidosome according to at least some embodiments of the invention.

FIG. 4 depicts brightfield optical micrographs of colloidosomes formed with polystyrene particles and stabilized with poly-L-lysine according to the method described in example 1. (a) shows colloidosomes formed from 1.3 μm diameter particles and (b) shows colloidosomes formed from 0.5 μm diameter particles. The colloidosomes in this figure have been transferred into water from a toluene/octanol solution as described in example 1.

FIG. 5 depicts brightfield optical micrographs of colloidosomes formed with polystyrene particles without stabilization in a toluene/octanol solvent as more fully described in example 1. (a) shows colloidosomes formed from 0.5 μm diameter particles and (b) shows colloidosomes formed from 1.0 μm diameter particles.

FIG. 6 depicts 3-dimensional confocal fluorescence images of colloidosomes formed with 0.7 μm polymethylmethacrylate beads in a water-in-oil system without stabilization as more fully described in example 2. Top, a 3-dimensional projection; Bottom, a 3-dimensional reconstruction.

FIG. 7 depicts scanning electron micrographs of a 10 μm diameter colloidosome formed from 0.9 μm diameter polystyrene spheres in 50 volume % vegetable oil and 50 volume % toluene. The colloidosomes have been dried after sintering at 105° C. for 5 minutes and interface removal as more fully described in example 4; (b) shows a 10 μm diameter colloidosome and (a) shows a close-up view of (b). The arrow in (a) points to one of the 0.15 μm pores that define the permeability.

FIG. 8 depicts micrographs of colloidosomes demonstrating their selective permeability. Colloidosomes formed from 0.9 μm diameter particles in 50 volume % vegetable oil and 50 volume % toluene in an aqueous solvent were subject to interface removal and were exposed to 0.5 μm and 0.1 μm probe particles in the exterior phase for 8 hours prior to recording the images in (a) and (b); (a) brightfield microscope image, the arrows point to larger probe particles that are excluded from the interior of the colloidosome; (b) a fluorescence micrograph, arrow points to smaller probe particles that can pass through the pores of the colloidosome and enter the chamber therein.

FIG. 9 depicts scanning electron micrographs of colloidosomes prepared with 0.9 μm polystyrene beads modified with aldehyde sulfate groups after sintering for various periods of time [0 minutes (upper left); 5 minutes (upper right); 20 minutes (lower left); and 2 hours (lower right)] as more fully described in example 4.

FIG. 10 depicts confocal fluorescence images of colloidsomes formed with polymethylmethacrylate in decalin and stabilized by swelling as described in example 5. Top, a top view of a colloidosome; Middle, an oblique view of a colloidosome; Bottom, a view of a broken colloidosome.

FIG. 11 is a brighffield optical micrograph of a multi-layered colloidosome formed with polystyrene (latex) beads functionalized with sulfate in dodecane/ethanol according to the method described in example 6.

FIG. 12 depicts a brightfield optical micrograph of colloidosomes formed with amidine-modified polystyrene beads as described in example 7. (a) top view of a colloidosome; (b) bottom view of the colloidosome in (a).

FIG. 13 depicts a colloidosome encapsulating a fibroblast cell. The colloidosome was formed from polymethylmethacrylate beads in decalin as more fully described in example 8.

FIG. 14 is a drawing that depicts a cross-section of a colloidosome having encapsulated therein a pancreatic cell that secretes insulin. As seen in the figure, antibodies are prevented from entering through the pores of the colloidosome whereas insulin can exit the colloidosome through the pores.

### DETAILED DESCRIPTION OF THE INVENTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention provides methods for making self-assembled, selectively permeable microscopic structures referred to herein as colloidosomes. The colloidosomes may advantageously be used, for example, for encapsulating desired active agents as more fully described herein. In at least some embodiments of the invention, a method includes providing particles formed from a biocompatible material in a first solvent and forming an emulsion by adding a first fluid to the first solvent, wherein the emulsion is defined by droplets of the first fluid surrounded by the first solvent. The emulsion may be an oil-in-water or a water-in-oil emulsion. The method includes coating the surface of the droplets with the particles and stabilizing the particles on the surface of the droplets to form a stable colloidosome that has a yield strength of at least about 20 Pascals. The colloidosomes formed include an outer layer, or shell, of the particles that define an internal enclosure, such as a chamber or cavity, and will be more fully described herein.

The method, in at least some embodiments, may include transferring the colloidosome into a second fluid and isolating or otherwise recovering substantially intact colloidosomes, wherein the second fluid is substantially identical to the first fluid, or alternatively, the second fluid is substantially different from the first solvent. By “substantially intact”, it is meant herein that at least about 80%, or at least about 90%, or at least about 95%, and even at least about 99% of the colloidosomes remain intact after removing the oil-water interface by transferring the colloidosomes from the, for example, first solvent into a second fluid substantially the same as the first fluid as described herein.

By “substantially identical”, it is meant herein that the fluids involved are chemically similar to each other and/or have similar solubility properties. Additionally, “substantially identical” fluids include fluids in which one can not observe separate phases if the fluids are mixed together and/or the fluids are otherwise miscible. As one example, the second fluid and the first fluid can be aqueous solvents. As a further example, the second fluid and the first fluid can be organic solvents.

By “substantially different”, it is meant herein that the fluids involved are not chemically similar to each other and/or do not have similar solubility properties. Additionally, “substantially different” fluids include fluids in which one can observe separate phases if the fluids are mixed together and/or the fluids are otherwise immiscible. As one example, the second fluid can be an organic solvent and the first fluid can be an aqueous solvent.

The methods advantageously form colloidosomes that have desired structural properties. For example, the colloidosomes are surprisingly able to withstand a large amount of yield stress. The formed structures may be advantageously used, for example, for encapsulating a desired active agent. Accordingly, in another aspect of the invention, methods for encapsulating desired active agents are also provided.

In at least some embodiments, a method for encapsulating a desired active agent includes providing particles formed from a biocompatible material in a first solvent and forming an emulsion by adding a first fluid, such as a solvent, containing an active agent to the first solvent wherein the emulsion is defined by droplets of the first fluid surrounded by the first solvent. The method includes coating the surface of the droplets with the particles and stabilizing the particles on the surface of the droplet to form stable colloidosomes having a yield strength of at least about 20 Pascals.

In yet another aspect of the invention, self-assembled, selectively-permeable colloidosomes are provided. In some embodiments, a colloidosome includes a shell formed of biocompatible, substantially spherical particles wherein each of the particles are linked to its neighboring particles. The outer shell defines an inner chamber and has a plurality of pores. In some embodiments, the chamber is sized for housing an active agent. In some embodiments, the colloidosome is non-biodegradeable, but may be biodegradeable upon selection of appropriate starting materials in selected circumstances as desired. Additionally, in at least some embodiments, the colloidosome has a yield strength of at least about 20 Pascals. In certain forms of the invention, the particles are linked to neighboring particles by van der Waals forces, or other electrostatic forces; chemical cross-linking of the particles, from coalescence of the particles in one or more regions of the particles or from a combination thereof.

In one aspect of the invention, methods for making self-assembled, selectively permeable structures referred to herein as colloidosomes are provided. In one form of the invention, a method includes providing particles formed from a biocompatible material in a first solvent and forming an emulsion by adding a first fluid to the first solvent, wherein the emulsion is defined by droplets of the first fluid surrounded by the first solvent. The method includes coating the surface of the droplets with the particles and stabilizing the particles on the surface of the droplets to form stable colloidosomes having a yield strength of at least about 20 Pascals. The method, in certain embodiments, includes transferring the colloidosomes from the first solvent into a second fluid substantially identical to the first fluid and recovering substantially intact colloidosomes as described herein.

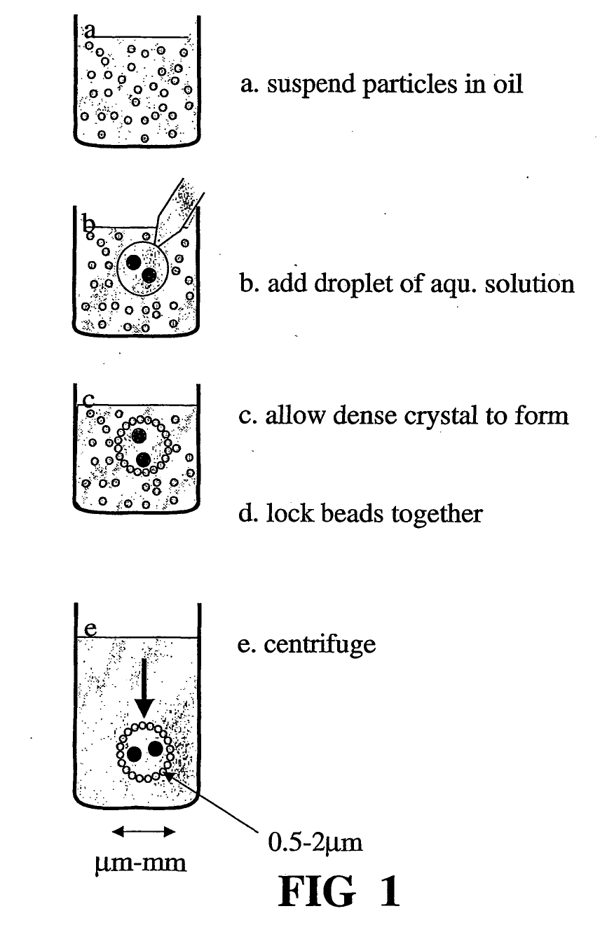

Referring now to FIG. 1, a fabrication method used to form colloidosomes in at least some embodiments of the invention is described. The method is described for a water-in-oil system, but may readily be used to obtain oil-in-water emulsions. As seen in the figure, colloidal particles are first suspended in oil. For clarity, only a single droplet of aqueous solution is shown being added to form an emulsion. The solution may be swirled or otherwise mixed slightly, if desired. However, high shear is not required to self-assemble the colloidosome. Beads are locked together as indicated in the figure by a swelling process or with use of a polyelectrolyte, such as a polycationic agent as more fully described herein. Other methods of stabilizing or otherwise locking the beads together are described herein. The colloidosomes are then isolated and subject to interface removal by a centrifugation process. It has been determined herein that at least about 100, or in other embodiments at least 1000, colloidosomes can be produced in a single test tube according to the methods described herein and it is expected that the process can be scaled to larger quantities.

The fluids, such as the solvents, utilized in the methods described herein are, in certain embodiments, liquids, such as organic solvents and aqueous solvents, although use of gaseous fluids is also envisioned as more fully described herein. The fluids are selected such that the fluid used to form the droplet and the fluid in which the droplet is placed to form the emulsion are immiscible. The choice of fluids selected will depend on the nature of the particles used to make the colloidosome, and the nature of the internal liquid phase of the colloidosome. For example, if a colloidosome with a cavity filled with an aqueous phase is desired, then the particles may be suspended in an organic solvent as the first solvent and the emulsion can be formed with water or other aqueous solution as the first fluid. If a colloidosome with a cavity filled with an organic phase is desired, then the particles may be suspended in an aqueous solvent as the first solvent and the emulsion may be formed with an organic solvent as the first fluid.

A wide variety of aqueous solvents may be utilized. Exemplary aqueous solvents include water, and liquids highly soluble in water, such as glycerol, ethylene glycol, formamide or similar solvents and combinations thereof. In at least some embodiments, the solvent includes water. Additionally, a wide variety of organic solvents may be utilized. Such organic solvents are generally water-immiscible fluids, or fluids that that, when combined, can form discrete interfaces. Organic solvents typically will dissolve only trace quantities of an aqueous solution, such as no more than about 0.0001 g to 0.001 g aqueous solution/g of solvent. As described herein, such organic solvents include various oils. Suitable organic solvents include hydrocarbons, including alkanes such as dodecane and hexadecane; aromatic hydrocarbons, including toluene and benzene; decalin, selected alcohols, such as octanol; silicon oil, vegetable oil or other natural oil or similar solvents, and combinations thereof.

The particles utilized in the methods are typically formed of biocompatible materials that can self-assemble at an oil-water interface. Use of the term “oil” herein includes organic solvents as described herein. The particles are, in certain forms of the invention, formed of hydrophilic or hydrophobic components or other materials, or combinations thereof. The terms “hydrophilic” and “hydrophobic” are used herein and are defined in the art to mean “water-loving” and “water-hating”, respectively. Thus, the term “hydrophilic component” denotes a material that has functional or other chemical groups which have a strong affinity for water compared to a hydrophobic group whereas the term “hydrophobic component” denotes a material that has functional or other chemical groups which have little or no affinity for water compared to a hydrophilic group as known in the art. Additionally, the components may be monomeric, but are polymeric in other embodiments.

Exemplary hydrophobic materials used to form the particles include polystyrene, polyalkylmethacrylates, such as polymethylmethacrylate, polyethylmethyacrylate, polybutylmethacrylate; polyalkylenes, including polyethylene and polypropylene; and inorganic materials such as ceramics and including silica, alumina, titania that are surface-functionalized to make them hydrophobic. Suitable hydrophilic materials used to form the particles include organic polymers that can be functionalized with hydrophilic groups; clay particles, such as disk-shaped particles; biological materials, including pollen grains, seeds, and virus particles that have been treated so as to be non-infective or to otherwise to not cause disease; and particles, including nanoparticles, composed of metallic, electrically semiconducting or insulating materials, including gold, cadmium sulfide, cadmium selenide, zinc sulfate and combinations thereof. The term “nanoparticles” as used herein refers to particles with diameters less than about 20 nm

In at least some embodiments of the invention, the materials used to form the particles are derivatized or otherwise modified with selected functional groups in order to, for example, decrease aggregation of the particles. For example, when hydrophobic polystyrene particles are suspended in an aqueous solvent, it may be desirable to introduce ionic functional groups in order to reduce or eliminate particle interaction that may lead to aggregation. Although not intending to be bound or limited by any theory, it is believed that introduction of ionic groups leads to sufficient repulsion of particles so that they will not associate to the point of forming agglomerates.

The functional groups may be anionic or cationic. Suitable anionic groups include, for example, carboxylate, sulfate, aldehyde sulfate, aldehyde amidine, aliphatic amines and other groups and combinations thereof. Suitable cationic groups include amine, amidine and combinations thereof. Moreover, in at least some embodiments of the invention, the particles are substantially spherical or some similar shape. Thus, at least about 90% of the particles, in other embodiments at least about 95%, and in yet other embodiments at least about 100% of the particles are spherical or otherwise in the form of a bead.

In certain forms of the invention, the emulsion is formed by adding or otherwise suspending a first fluid in the first solvent. The first fluid is in the form of small drops, or droplets, in certain forms of the invention and is substantially immiscible in the first solvent. The droplets may be formed by adding the first fluid to the first solvent and gently agitating the container in which the first solvent is contained. Such a process also accelerates the self-assembly process. As this may generate some shear stress on the system, in some embodiments the droplets are formed with little or no shear stress during the self-assembly process by use of a pipet or by injecting the droplets into the solution with conventional droplet-forming machines known to the art. If the fluid is a gas, then the size of the gas droplets may be similarly controlled by appropriate modification of the convention machine described herein. As known in the art, shear stress may be determined by measuring the solvent velocity gradient and the solvent viscosity as known in the art and multiplying these values together.

The size of the colloidosomes formed in the method depends primarily on the size of the template emulsion droplet and the diameter of the particles utilized. In at least some embodiments, the droplet may range from, for example, about 50 nm to about 1000 μm, or about 10 μm to about 300 μm. Thus, the colloidosomes can similarly range in size from about 50 nm to about 1000 μm, and about 10 μm to about 300 μm, depending on the thickness of the colloidosome shell. It is realized that the structural integrity of the colloidosome decreases as a function of increasing diameter and should be taken into account when forming such structures.

In the process of coating the surface of the droplets with the particles, the particles self-assemble at the interface between the two fluids. Although not intending to be bound by any theory, it is believed that self-assembly of the particles, such as the beaded particles described herein, is driven by the minimization of total interfacial energy and whether they self-assemble is determined by the three interfacial energies (i.e., oil/water, oil/particle and water/particle) as discussed in, for example, Pieranski (1980) Physics Rev. Lett. 45:569-572. In at least some embodiments, at least about 90%, or at least about 95%, or even at least about 99% of the surface area of the droplets are covered with the particles.

After the surface of the droplets have been coated with the particles, the colloidosomes may then be stabilized in a variety of ways. For example, the particles may be linked to each other by van der Waals forces or other electrostatic interactions, with use of chemical cross-linking agents for inter-particle cross-linking, by a swelling process or by a sintering process. The latter processes can lead to physical linking or attachment of the particles to each other.

In at least some embodiments of the invention, the particles are linked by cross-linking between reactive surfaces of adjacent beads. In order to physically link each of the particles with its neighboring particles utilizing cross-linking agents, the cross-linking agent is added to the first solvent after the colloidosome is formed. A wide variety of cross-linking agents may be used, including dicyclohexylcarbodiimide (DCC), or other similar cross-linking agents known to the art, and combinations thereof.

In some embodiments of the invention, the particles are linked by mechanically locking adjacent beads. This is accomplished by forming bridge, or necks, between beads. In stabilizing the colloidosomes, or otherwise increasing the structural integrity or rigidity of the colloidosomes, by the sintering process, the solvent-suspended colloidosomes are incubated in an oven at the glass transition temperature (T_{g}) of the polymer that the particle is formed of for a period of time sufficient to at least partly coalesce the particles or otherwise merge or join the particles to increase the structural integrity of the colloidosomes. If T_{g} is higher than the boiling point of the solvent, the boiling point of the solvent may be increased by addition of solutes known to the art to increase the boiling point. For example, for an aqueous solution, glycerol, ethylene glycol, or other known solution or composition that increases the boiling temperature of an aqueous solution, or a combination thereof, may be utilized to increase the boiling point of the solution. As a further example, for an organic solvent, other organic solvents having a higher boiling point may be added to the organic solvent utilized to increase the boiling point of the solution.

During the sintering process, the particles may at least partly coalesce and linkages or “necks” between neighboring particles may be formed. Therefore, by “partly coalesce”, it is meant herein that a region of one particle and an opposing region of a neighboring particle will melt and mix together such that a continuous linkage or other bridge between the particles is formed and remains after the sintering process is completed and the particles have cooled to their initial temperature prior to the process, such as room temperature. In other embodiments, deformation of the beads can increase the bead-bead contact area, making the attractive force between the beads stronger without coalescence.

The time period for the sintering process should be selected such that complete coalescence does not occur whereby a non-porous shell is formed, unless such complete coalescence, and colloidosomes without pores, are desired. Although this time period may vary depending on the nature of the colloidosome and components and solvents utilized to form the colloidosomes, in some embodiments of the invention the colloidosomes are heated for a period of about 2 minutes to about 120 minutes, or no more than about 5 minutes.

In at least one embodiment of the invention, the structure of the colloidosomes is stabilized using a swelling method. In such a method, the colloidal particles can be at least partially coalesced to form a structurally stronger shell, or may otherwise exhibit increased interparticle attraction. In one form of a swelling method, in the case wherein the first solvent is organic, one or more organic solvents in which the colloidal particles are soluble in are added to the first solvent to otherwise contact the particles for a period of time sufficient for a region of the particles to at least partially solubilize and thereby at least partially coalesce with a region of its neighboring colloidal particles. As an example, when the colloidal particles are formed of polystyrene, an appropriate organic solvent includes toluene. As another example, when the colloidal particles are formed of polymethylmethacrylate, an appropriate organic solvent includes a combination of chlorobenzene and decalin in a volume ratio of about 35:65. Other suitable organic solvents may be determined by the skilled artisan taking into account the nature of the colloidal particle. The time required to stabilize the particles with the use of organic solvents described herein will vary with the nature of the solvents and the particles utilized. Generally, the amount of time the solvents contact the particles is about 1 minute to about 10 minutes.

In some embodiments of the invention, the colloidosomes are stabilized by utilizing one or more polyelectrolytes. The polyelectrolyte can be added to the solvent which is emulsified or may be added to the first solvent that includes the, for example, beaded particles. The nature of the polyelectrolyte will depend on the nature of the charge on the surface of the colloidal particles. Thus, polycationic agents can be utilized when the net charge on the surface of the colloidal particle is predominantly negative or when only negatively charged functional groups are on the surface, and polyanionic agents can be utilized when the net charge on the surface of the colloidal particle is predominantly positive or when the surface of the colloidal particles includes only positively charged functional groups. Exemplary polycationic agents include polyamino acids, including poly-L-lysine; poly(diallyldimethylammonium chloride)(PDMAC), poly(allylamine hydrochloride) or other similar polycationic agents or combinations thereof. Suitable polyanionic agents include, for example, poly(styrene sulfonate), including poly(sodium 4-styrenesulfonate); or other suitable agents or combinations thereof. Although not intending to be bound by any particular theory, it is believed that the polyelectrolyte forms a film that connects or otherwise stabilizes the particles.

In other forms of the invention, the surface of the particles is modified in order to increase the interaction of the polyelectrolyte with the surface of the particles. The surface of the particles can be modified utilizing various ionic functional groups as previously described. Additionally, the surface can be modified with other agents that will bind to another agent that may be added to the first fluid. For example, the particles can be modified with biotin and avidin can be added to the system. Other such combinations of agents include, for example, biotin and streptavidin.

After the colloidosomes are stabilized, in at least some embodiments, the colloidosomes are isolated in a variety of ways. In one embodiment of the invention, the interface is removed and the colloidosomes are isolated by use of centrifugal force, such as by transferring colloidosomes that are suspended in an organic solvent into an aqueous solvent wherein the chamber of the colloidosomes is filled with an aqueous solvent, or vice versa. If the colloidosomes are being transferred from an organic solvent into an aqueous solvent, for example, aliquots of the colloidosomes are placed on the top of the desired aqueous solution, which can include a non-ionic surfactant such as, for example, Tween, SPAN, Triton or other suitable non-ionic surfactant. If the colloidosomes are being transferred from an aqueous solvent into an organic solvent, such as where the internal chamber of the colloidosome is filled with an organic solvent, the colloidosomes are placed on the top of the desired organic solvent which has a density greater than the density of the aqueous solvent, but less than the density of organic solvent in the chamber of the colloidosome. The colloidosomes are then centrifuged at a centrifugal force and for a period of time sufficient for isolation. Although this time period may vary depending on the circumstances, the colloidosomes can be centrifuged at about 2000 g to about 14000 g for about 5 minutes to about 30 minutes. Typically, the colloidosomes can be centrifuged at about 9300 g for about 10 minutes in order to remove the colloidosomes from the oil-water interface. Other methods of isolating the colloidosomes herein include drying. The drying process is performed by soaking the colloidosomes in ethanol to remove the interface and then allowing the ethanol to evaporate. This is an effective drying process when both solvents used are miscible in ethanol. Other methods include use of organic solvents and extraction procedures.

The colloidosomes formed have advantageous mechanical properties. For example, not only are the colloidosomes elastic, they have a yield strength of about 20 Pascals to about 100 Pascals, or about 20 Pascals to about 500 Pascals, or about 20 Pascals to about 1000 Pascals and even at least about 20 Pascals to about 2000 Pascals. Additionally, the colloidosomes have a yield strength of at least about 20 Pascals, or at least about 50 Pascals, or at least about 100 Pascals, or at least about 200 Pascals, or at least about 500 Pascals, or at least about 750 Pascals and even at least about 1000 Pascals. The yield strength can be determined by using a cantilever to mechanically deform the colloidosome with a known stress and observing the response.

It is noted here that, depending on the circumstances, one may wish to have colloidosomes which release their contents upon exposure to an applied force. For example, it a particular chemical is encapsulated in a food product, and the chemical is to be released by applying pressure, such as by chewing, the yield strength of the colloidosomes is selected to allow capsule destruction during chewing to be no more than about 10 MegaPascals. In other embodiments, the yield strength of the colloidosomes can be more than about 1 MegaPascals Alternatively, the colloidosomes formed are structurally stable or otherwise have sufficient structural integrity so that at least about 80%, or at least about 90%, or at least about 95%, and even at least about 99% of the colloidosomes remain intact after removing the oil-water interface as described herein. The interface can be removed as noted herein by, for example, transferring the colloidosomes from the first solvent into a second solvent substantially identical to the first fluid or by other methods described herein that do not substantially affect the structural stability of the colloidosomes, or otherwise damage the colloidosomes. The number of colloidosomes that remain intact after the transfer is typically determined by inspection utilizing an optical microscope.

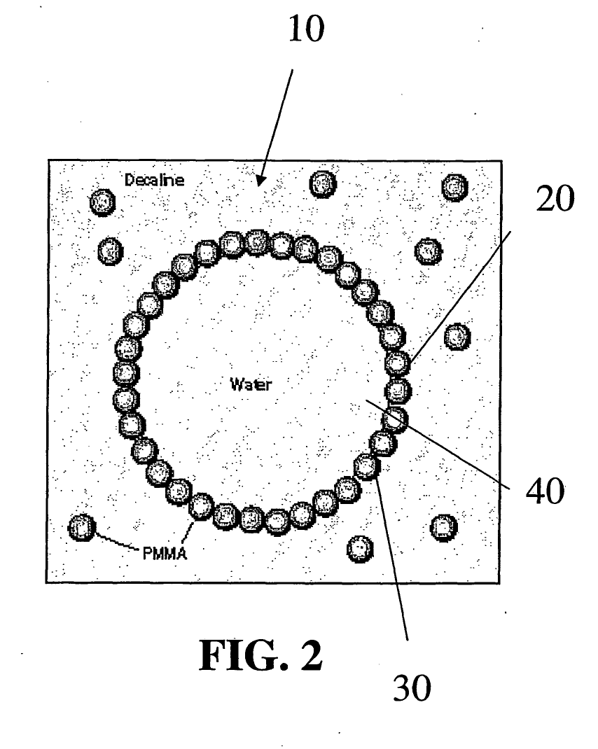

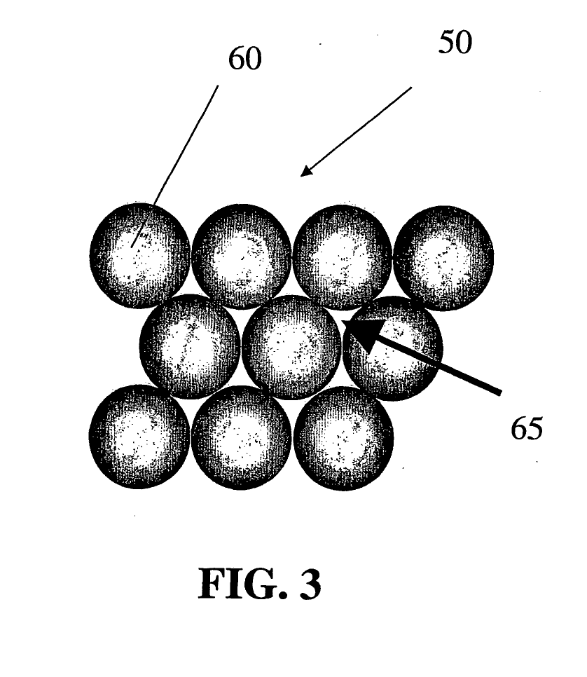

Referring now to FIG. 2, a drawing of a cross-section of a colloidosome formed with polymethylmethacrylate (PMMA) colloidal particles utilizing an aqueous solution in decalin oil according to the methods described herein is shown. It can be seen that colloidosome 10 includes shell 20 formed of a monolayer of particles 30 that defines inner chamber 40. FIG. 3 depicts a drawing showing a cross-section of a colloidosome 50 that includes close-packed spheres 60 and interstitial pores 65. The pore size may be controlled by, for example, the size of the particles utilized to form the colloidosome. For example, use of beaded particles of larger diameter lead to larger pore sizes whereas use of beads of smaller diameter lead to smaller pore sizes. Additionally, a mix of both smaller particles and larger particles can be used in forming a colloidosome to achieve a smaller pore size while retaining the advantageous properties of colloidosomes made with only larger particles.

In another aspect of the invention, methods of encapsulating an active agent utilizing the colloidosomes formed herein are provided. The colloidosomes can advantageously be used, for example, for controlled release of the active agents. In one form, a method includes providing particles formed from biocompatible materials in a first solvent and forming an emulsion by adding a first fluid, such as a solvent, containing the active agent to the first solvent wherein the emulsion is defined by droplets of the first fluid surrounded by the first solvent. The method includes coating the surface of the droplets with the particles and stabilizing the-particles on the surface of the droplet to form a colloidosome having encapsulated therein the desired active agent. The colloidosomes formed have a yield strength of at least about 20 Pascals or otherwise as described above. The method, in certain embodiments, includes transferring the colloidosomes from the first solvent into a second fluid substantially identical to the first fluid and recovering substantially intact colloidosomes as described herein.

In certain forms of the invention, the particles utilized in the methods of encapsulating an active agent are also substantially spherical and can be formed of biocompatible polymers as described herein. Thus, as can be seen, one form of the method of encapsulation is similar to the methods of forming the colloidosomes described herein with the exception that the first fluid is a solvent that includes or otherwise contains the desired active agent. Additionally, the first fluid can be selected based on the nature of the active agent, and may be a liquid or a gas. Therefore, the first fluid can be chosen such that it can solubilize the active agent or will otherwise be compatible with the active agent. For example, if the active agent is a hydrophobic material, such as certain drugs, then methods would include utilizing an oil-in-water emulsion such that the first solvent is aqueous and the first fluid is an organic solvent or oil. If the active agent is a hydrophilic material, such as some biological macromolecules, or is a material that is otherwise compatible with aqueous solutions, such as biological cells, then a water-in-oil system is used wherein the first solvent is an organic solvent or oil and the first fluid is an aqueous solution.

A wide variety of active agents may be encapsulated according to the methods described herein. “Active agent”, as used herein, refers to an agent that has a beneficial effect in a biological system, such as in or on the body of a patient, or otherwise provides advantages when added or otherwise applied to a system as described herein. The active agent can be, for example, a biological agent or a chemical agent. Chemical agents include, for example, drugs or other pharmaceutical agents, flavoring agents or chemicals that give rise to fragrances. The biological agents include, for example, biological macromolecules, such as proteins, nucleic acids, such as deoxyribonucleic acid (DNA) or ribonucleic acid (RNA) that can encode, for example, a desired protein; vitamins, fats or other lipids; carbohydrates, and combinations thereof to form various food products. The active agent can also be a gas, such that when the colloidosomes are added to, or are formed in, such a system in which foams are formed, the foams are stabilized. In certain embodiments, the foams formed in this manner are opaque from the scattering of light by the particles that are present on the gas droplet surfaces. This can be advantageous when applied to foods, as opaque foams are more aesthetically pleasing.

In use, the active agents can diffuse out of the colloidosome through the pores if the active agent is sized to fit through the pores. If it is desired that such active agents whose size is larger than the pore size be released from the capsule in use, a wide variety of methods are available. For example, it has been determined herein that the contents can be released by rupture if sufficient shear is applied, or by application of compressive stresses. Additionally, because the fabrication process depends only on the surface properties of the colloidal particles, there is substantial freedom to choose the material in the core of the particles to add functionality. In at least some embodiments, a portion of the particles may be made of a material that increases its volume, for example, upon increasing the pH as in alkali-swellable microgel particles. The swelling of some particles is likely to introduce substantial surface stresses that would tear holes in the capsule that would allow release of the contents. Alternatively, some of the particles could be made from a material easily dissolved in situ (chemically or photochemically), thus creating large holes in the capsule and releasing the contents.

In yet another aspect of the invention, colloidosomes, or capsules, are provided. In one form, a colloidosome includes a shell formed of biocompatible, substantially spherical particles wherein each of the particles are linked to a neighboring particle, typically each of its neighboring particles. The shell is an outer layer that defines an inner chamber or cavity and has a plurality of pores extending therethrough. The chamber is sized to house or otherwise contain an active agent as described herein. In certain embodiments, the shell is formed of a monolayer of the spherical particles, although multi-layer shells are also envisioned as more fully described below. The colloidosomes are quite strong, having the preferred yield strengths as described above. Additionally, the colloidosomes can withstand relatively high yield shear rates. For example, the colloidosomes, such as those having a diameter of about 10 μm to about 50 μm in water, have a yield shear rate of at least about 10s⁻¹, or at least about 25 s⁻¹, or at least about 50 s⁻¹, even at least about 75 s⁻¹ and even at least about 100 s⁻¹. Such yield strengths may further be greater than about 100 s⁻¹ in certain forms of the invention. The nature of the spherical particles or other components of the colloidosomes has already been described above. The colloidosomes can be substantially spherical, elliptical or other rounded shape. As one example, the aspect ratio of the colloidosome can be about 2:1.

The thickness of the outer shell, or layer, is dependent on the diameters of the particles utilized to form the colloidosome and the number of layers present. In certain forms of the invention, the shell is an outer layer that is a monolayer of the particles and thus the thickness of the shell can range from about 20 nm to about 20 μm, or about 100 nm to about 10 μm, or about 0.5 μm to about 1 μm. Additionally, the shell may be formed from multiple layers of the particles, including two, three, four or more layers, and thus the diameter of the outer layer can be two, three, four or more times the diameters mentioned above. Such multiple layers can be formed, for example, by allowing aggregation of the particles when suspended in the first solvent as described above.

The outer shell defines an enclosure, such as a chamber or cavity that may advantageously be utilized to house or otherwise contain an active agent as described herein. The size of the chamber is dependent on the size of the emulsion droplet template, and can thus be varied accordingly as described herein. As described above, the droplet, and thus the diameter of the chamber, can range in size from, for example, 50 nm to about 1000 μm, or about 10 μm to about 300 μm. The size of the chamber is chosen depending on the application. For example, if the colloidosome is to deliver a biological cell which, in certain forms of the invention, secretes a desirable substance, such as a pancreatic cell secreting insulin, the chamber is sized to accommodate the cell and is, for example, at least about 10 μm in diameter. As further described above, the diameter of the colloidosome can be about 50 nm to about 1000 μm or about 10 μm to about 300 μm. Additionally, at least about 50% of the colloidosomes, further at least about 60%, or at least about 70%, or at least about 80%, or at least about 90% and even at least about 95% of the colloidosomes have a diameter of about 50 μm to about 200 μm or can be greater than at least about 50 μm.

The colloidosomes have well-defined pores whose size can be varied depending on the application. For example, if a colloidosome has encapsulated therein a biological cell, the pores are large enough to allow any desirable substance produced by the cell to diffuse out of the chamber through the pores and external to the colloidosome, as well as allow desirable substances necessary to sustain the cell, such as glucose or other nutrients, to enter the chamber. It is realized that the pores for such an application are sufficiently small or otherwise sized to prevent entry into the chamber by immune system cells or immune system components, such as various antibodies, as well as to prevent the encapsulated cell from exiting the chamber through the pores. As previously described herein, the pore size can be adjusted by the size of the particles utilized. For example, use of beaded particles of larger diameter lead to larger pore sizes whereas use of beads of smaller diameter lead to smaller pore sizes. Thus, appropriate outer layer particles are chosen to form pores of the desired size. Although pore size can vary depending on the application, pore sizes can range from about 3 nm to about 3 μm, about 10 nm to about 1000 nm, or about 75 nm to about 200 nm. When encapsulating a biological cell, pore sizes are typically no more than about 1 μm to about 3 μm.

In certain embodiments of the invention, the pore sizes in a colloidosome are substantially uniform. That is, at least about 90%, or about 95%, or even about 100% of the pores of the colloidosome are of the same size and may, for example, have the same radius and thus the same diameter. In other forms of the invention, the radius of the pores may differ by about 50% to about 300%, resulting in pores differing in diameter by up to a factor of about 1.5, or even by a factor up to about 4. Alternatively, the pores may differ in radius by up to about 50%.

In other embodiments of the invention, the colloidosomes described herein are be included in a suspension. The colloidosome suspension includes, in at least one embodiment, a colloidosome suspended in a first solvent wherein the colloidosome has a shell formed of biocompatible, substantially spherical particles. Each of the particles are linked to neighboring particles as previously described. The shell defines an inner chamber and has a plurality of pores extending therethrough. The chamber is sized for housing an active agent and filled with a second solvent that is substantially identical to the first solvent. As one example, the chamber is filled with an aqueous solution and the solvent that the colloidosome is suspended in (i.e., the exterior solvent) may be the same or a similar aqueous solution. Such solvents, as well as other solvents, have been previously described herein.

Reference will now be made to specific examples illustrating the compositions and methods above. It is to be understood that the examples are provided to illustrate preferred embodiments and that no limitation to the scope of the invention is intended thereby. The following materials apply to the examples wherein polystyrene beads were used.

### Materials

The suspensions of polystyrene beads were obtained from Interfacial Dynamics Corporation (IDC). Divinylbenzene crosslinked beads 1.3 and 0.5 μm in diameter with carboxyl surface charge groups (DVB carboxyl beads) were used, along with biotin-coated 0.9 μm-diameter beads with aldehyde sulfate surface charge groups (aldehyde sulfate beads). Prior to being used, the contents of the bottle were redispersed by vortexing for a few seconds and then cleaned as described in the following Methods section. The carboxyl-modified fluorescent polystyrene probe particles (1.0 μm-diameter, excitation/emission wavelengths of 580/605 nm; 0.5 μm, 580/605; 0.1 μm, 505/515 nm) were provided by Molecular Probes.

The 1-octanol, toluene, dodecane, glycerol (all 99% pure), dimethyidichlorosilane, TWEEN20, and SPAN80 were purchased from Aldrich and not subject to further purification before use. The silicone oil (Fluka), ethanol (200 proof, Pharmco), acetone (Baker) and poly-L-lysine 0.1% w/v aqueous solution (Sigma, P8920) were also used as obtained from the manufacturers. the deionized water (Dl) used for the experiments was purified by a Millipore Milli-Q system. Wesson vegetable oil was filtered with a 0.45 μm-pore hydrophobic syringe filter prior to use.

### EXAMPLE 1

### Formation of Colloidosomes Utilizing Polystyrene Colloidal Particles and Stabilization with Poly-L-Lysine

### Protocol

Polystyrene beads cross-linked with divinyl-benzene (DVB-PS; 0.5 μm and 1.3 μm diameter) and carboxylate-modified were obtained from Interfacial Dynamics, Portland, Oreg. The internal cross-linking with divinyl benzene prevents dissolution of the particles in toluene. The beads were suspended in a solution of 90 volume % toluene and 10 volume % octanol at a volume fraction of about 10⁻³. About 10 μl of aqueous solution was added per ml of toluene solution and the solution was vortexed to break the water droplets (mean droplet diameter ranged from 50 μm to 500 μm) and to accelerate particle adsorption. At this stage, the DVB-PS beads diffused on the surface about their lattice positions.

The beads were then locked together to form a strong shell that remains intact after the water-oil interface is removed. In order to lock the beads together, the above procedure was repeated, except that the aqueous phase was a 1 mg/ml polycationic poly-L-lysine (150-300 kD) solution.

The exterior phase was then replaced with water. The capsules were washed in octanol and approximately 0.1 ml of the octanol-capsule solution was added to the top of 1 ml of an aqueous solution of non-ionic surfactant (10 mg/ml of Tween 20). The capsules were centrifuged at 9300 g for 10 minutes.

The permeability of the colloidosomes to probe colloidal particles of various sizes was then quantified. The colloidosomes were suspended in water containing polystyrene spheres of various sizes. Individual colloidosomes were inspected in an optical microscope and the number and sizes of particles within the colloidosomes was determined.

### Analysis

After adding the droplets of aqueous solution into the toluene/octanol solution, an ordered, complete monolayer of collioidal spheres was observed. Interactions among the particles at the interface were measured by tracking the particle velocities at various displacement, or by obtaining a probability density as a function of interparticle separation. The result is an interparticle potential. The potential provides information on how the particles will interact with each other as a function of interparticle separation. A long-range electrostatic repulsion was found, perhaps due to the dissociation of charges on the hydrated surfaces of the particles. The repulsion stabilizes the particles and forces them spontaneously to adopt a stable, densely-packed crystal on the spherical surface. Despite the significant repulsion between adsorbed particles (several k_{B}T), the particles densely covered the surface due to their deep surface-energy minimum (about 10⁵ to 10⁶ kT) that holds the particles to the surface. The particles are therefore a realization of a two-dimensional fluid of replusive disks which is known to exhibit a crystallization transition.

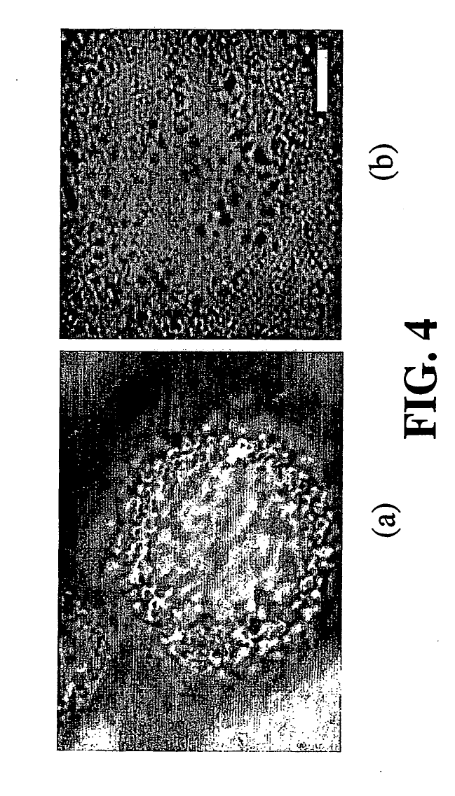

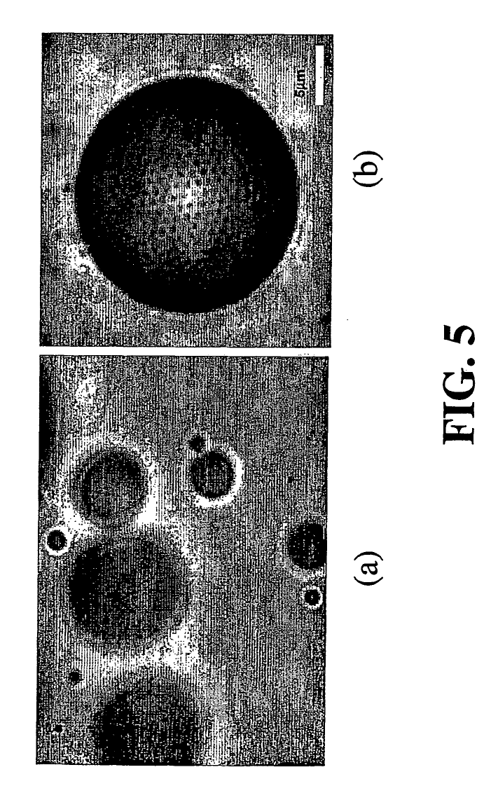

After self-assembly of the colloidal particles, the DVB-PS beads diffused on the surface about their lattice positions. Such colloidosomes are shown in FIG. 5, wherein (a) and (b) represents a brighffield optical micrographs (Leica DMIRB inverted microscope) taken with a Hamamatsu Orca-ER (C4742-95) digital camera. In reference to FIG. 5, (a) represents images of colloidosomes formed from 0.5 μm diameter particles and (b) represents images of colloidosomes formed from 1.0 μm diameter particles. After treatment with poly-L-lysine, (which adsorbed to the interior aqueous surface of the particles as verified using fluorescence labeling), the beads were locked together so that they no longer diffused on the surface. FIG. 4 shows brightfield optical micrographs of colloidosome formed as described herein with 1.3 μm polystyrene beads as described herein. The image in (a) depicts colloidosomes formed utilizing 1.3 μm diameter beads whereas the image in (b) depicts colloidosomes formed from 0.5 μm diameter particles. The image was taken by optical microscopy as described herein and known to the art.

Assuming the probe particles can penetrate the shell, an estimate of the time required for entry of probe particles—assuming they can penetrate the shell—is (1+πR/Ns)/(4πDRc) (about a few seconds; 25φ[s] for 1-μm beads) where D and c are the probe particles' diffusion coefficient and concentration, R is the probe particle radius, N is the number of pores and s is the radius of the pores, which are here approximated as circles. After more than 100 times the estimated entry time, no further change in density of probe particles inside or outside the colloidosomes was observed. The colloidosomes prepared with 1.3 μm beads were systematically impermeable to 1 μm diameter beads, but allowed 0.1 μm beads to penetrate freely.

### EXAMPLE 2

### Formation of Colloidosomes Utilizing Polymethylmethacrylate Colloidal Particles without Stabilization

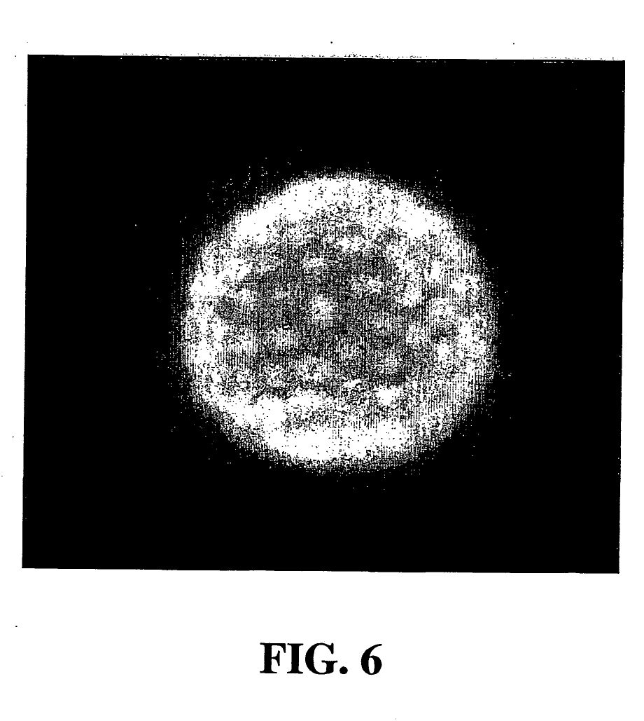

In this example, colloidosomes were formed as in example 1, with the exception that, instead of polystyrene beads in toluene/octanol, polymethylmethacrylate (PMMA) beads (0.7 μm diameter) were suspended in decahydronapthalene (decalin) and no poly-L-lysine, or other stabilizing agent, was used for stabilization. The colloidosomes were not transferred into water in this example. In this system an ordered, complete monolayer of colloidal spheres was also observed as seen in the fluorescence confocal microscope image seen in FIG. 6.

### EXAMPLE 3

### Formation of Colloidosomes Utilizing Functionalized Polystyrene Colloidal Particles Without Stabilization

In this example, polystyrene spheres (0.9 μm diameter functionalized with biotin, from Interfacial Dynamics, Portland, Oreg.) were suspended in water at a volume fraction of 10⁻³. About 10 μl of silicon oil droplets per ml of water was added to the water to form an emulsion. Using these methods, the polystyrene beads assembled at the surface of the oil droplets as discussed in the preceding examples, except that the particles surprisingly adhered to one another at the surface. The beads were unexpectedly stable in the aqueous solution and it was therefore not necessary to add any stabilization agent to the oil phase to lock the beads together at the surface. The external water phase was replaced with oil by placing a 0.1 ml aliquot of the aqueous solution with coated droplets in a vial on top of 1 ml of dense silicone oil and the colloidosomes were centrifuged at 9300 g for 10 minutes.

Although not intending to be bound by an particular theory, it is believed that the stabilization arises from the enhanced interparticle attraction provided by the silicon oil due to diminished electrostatic repulsion.

### EXAMPLE 4

### Formation of Colloidosomes Utilizing Polystyrene Colloidal Particles and Stabilization by Sintering

In this example, 0.9 μm polystyrene spheres that were biotinylated and functionalized with aldehyde sulfate were added to water at a volume fraction of about 10⁻³. Droplets of a solution of 50 volume % filtered vegetable oil (Wesson) and 50 volume % toluene were added to the water and the beads self-assembled at the oil-water interface. In order to lock the beads together, a sintering process was utilized. In this case, 50 volume % glycerol was added to the exterior aqueous phase to increase the boiling point of the solution prior to exposing the solution to a temperature of 105° C. for about five minutes. The polystyrene particles coalesced slightly, creating 150 nm necks between them. The shell therefore contained a continuous polystyrene shell with a regular array of holes.

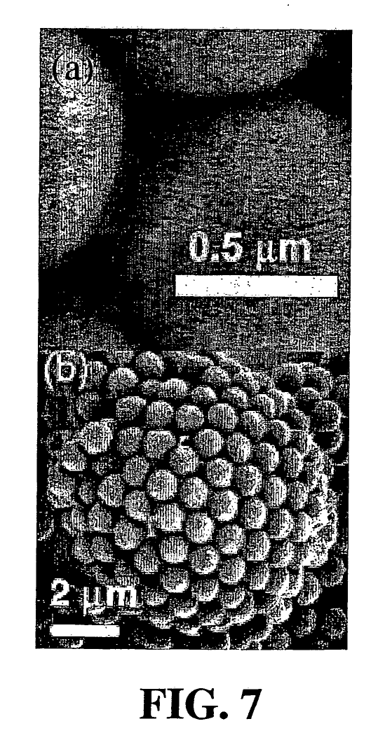

The colloidosomes were washed with ethanol and dried in a vacuum so they could be viewed under an electron microscope. Scanning electron micrographs of a dried collidosome prepared by this method are shown in FIG. 7. It was found that sintering the particles for longer times had an effect on the pore size and, it is believed, the strength of the capsule. For example, after sintering the particles for 20 minutes, the particles coalesced completely and the holes were completely filled.

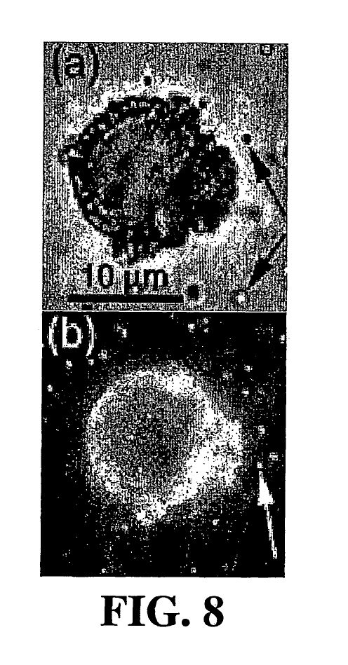

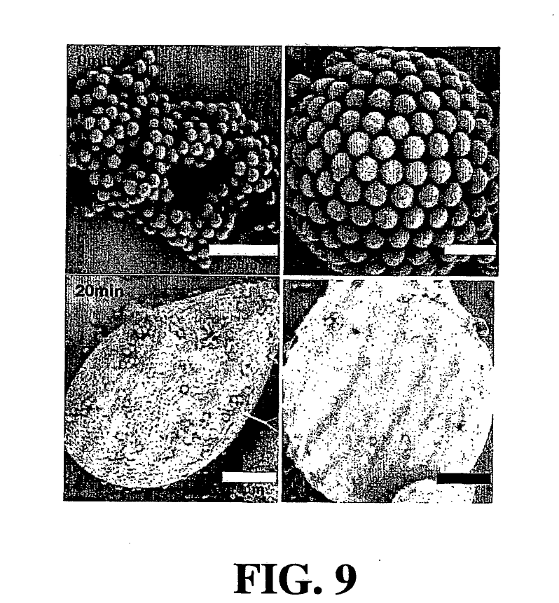

FIG. 8 depicts microscope images of colloidosomes used in determining the permeability of the colloidosomes. The colloidosome were formed as described above for the colloidosome in FIG. 7 but were not dried prior to analysis. The image in (a) is a brighffield microscope image showing that the larger probe particles (i.e., 0.5 μm diameter) denoted by the arrow are excluded from the interior of the colloidosome. Note that diffraction from the particles that form the colloidosome shell is faintly visible. The image in (b) is a fluorescence image showing that smaller probe particles (i.e., 0.1 μm diameter) as indicated by the arrow are able to pass through the pores and into the interior of the colloidosome. It was determined by the method described in example 1 that colloidosomes prepared by this method after sintering for 5 minutes were impermeable to 0.5 μm diameter probe particles but were permeable to 0.1 μm diameter probe particles. The effect of sintering time on colloidosomes formed with 0.9 μm biotin-coated polystyrene beads with aldehyde sulfate surface charge groups after drying is shown in FIG. 9. The colloidosomes prepared with polystyrene functionalized with aldehyde sulfate and biotinylated were sintered for a period of 5 minutes, 20 minutes and 2 hours and scanning electron micrographs were taken. After sintering, the colloidosomes were soaked in ethanol for 24 hours and allowed to dry in air for 24 hours. As seen in FIG. 9, unsintered colloidosomes (upper left corner micrograph) were unstable to drying but become stable after sintering for 5 minutes (upper right corner micrograph). Interstitial pores are also prominent, but gradually disappear as the sintering process continues as seen in the in the figure at the 20 minute and 2 hour time intervals (bottom left and right micrograph, respectively).

### EXAMPLE 5

### Formation of Colloidosomes Utilizing Polymethylmethacrylate Colloidal Particles and Stabilization by Swelling

Polymethylmethacrylate (PMMA) particles (0.03 ml of 10-20 volume % PMMA in decalin) (provided by Andrew Schofield, University of Edinburgh), were suspended in 0.3 ml decalin. The volume fraction of PMMA particles in this mixture was about 1-2%. About 1 to 10 microliters of water (first fluid) was then added (the water can include 0.1M NaCl and/or fluorescein dye. The dye is for aiding visualization of the particles). The sample was then shaken to produce small (20-300 micron) water drops. Alteratively, the water phase can be added as one large droplet (about 400 microns).

Chlorobenzene was then added to the decalin containing the coated droplets in an amount of 0.2 ml. Although not intending to be limited by any particular theory, it is believed that this step swells the particles, since chlorobenzene is a good solvent for PMMA. Within about 5 minutes, about 0.1 mL of the above chlorobenzene/decalin/PMMA/water solution was added to 3 mL of a mixture of 50 volume % toluene and 50 volume % decalin.

Within about 5 minutes, the coated droplets were transferred to 4 ml of decalin by withdrawing the droplets in a pipette and injecting them into a vial with decalin in order to wash away toluene. The resulting coated droplets are stable against coalescence and it is believed they can be stored in decalin indefinitely.

### Results

Inspection of samples approximately 5 minutes after addition of water to the PMMA suspension showed spherical water droplets fully, densely covered with PMMA beads. The beads were well ordered on the surface (in a two-dimensional hexagonal lattice). FIG. 6 depicts a colloidosomes that is representative of this particular stage in colloidosome formation as it has not yet been stabilized.

After adding the chlorobenzene/decalin/PMMA/water solution to the toluene/decalin solution, microscope inspection at this stage revealed fully coated droplets, very much like that found prior to the swelling procedure, with the exception that some of the droplets are highly non-spherical (e.g. aspect ratios of 2:1), indicating that the PMMA layer has some elasticity.

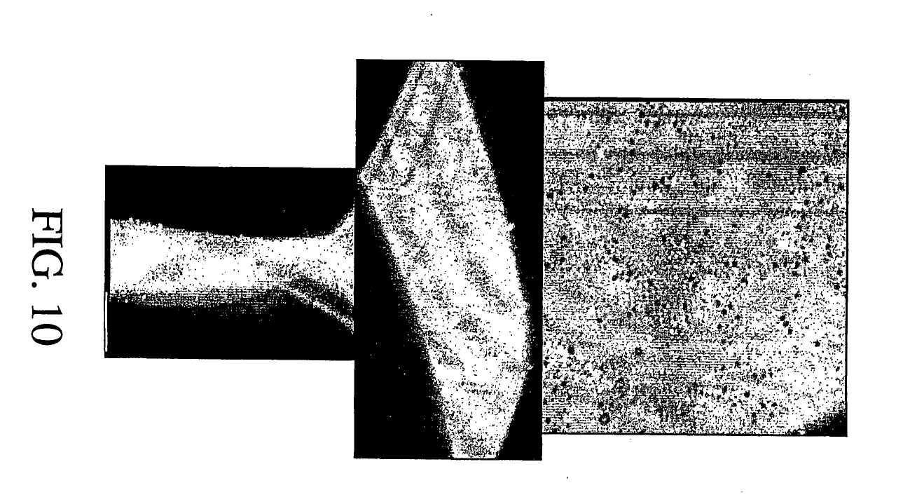

FIG. 10 depicts confocal fluorescence images taken of the colloidosomes formed according to the procedure outlined in this example. As seen in FIG. 10, bottom, a colloidosomes wherein the droplet has ruptured is shown. The two-dimensional rafts of PMMA particles are stuck to one another as seen in the FIG. 10, bottom, thus providing evidence that the stabilization process worked.

### EXAMPLE 6

### Formation of Multilayer Colloidosomes Utilizing Functionalized Polystyrene Colloidal Particles

### Protocol

In this example, colloidosomes were formed as in example 1, with the exception that, instead of carboxylate-modified (DVB-PS) in toluene/octanol, 1.0 micron beads with sulfate groups were suspended in dodecane (90 volume %) and ethanol (10 volume %), also at a volume fraction of 10⁻³. About 10 microliters of water were added per ml of dodecane/ethanol solution and the solution was vortexed to break the water droplets (mean droplet diameter ranged from 50 μm to 500 μm) and to accelerate particle adsorption.

### Results

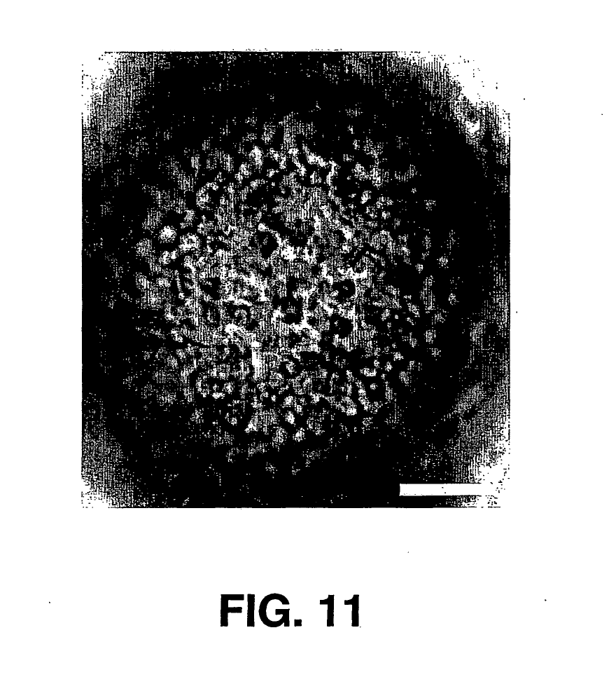

Instead of monolayer shells of particles, shells of aggregates of beads (or equivalently, colloidosome shells with multilayers of particles) were obtained. Because particles aggregate in dodecane (due to the absence of long-range electrostatic repulsion), the particles of the shell which are completely in dodecane/ethanol are already stuck together and there is thus no need for additional stabilization. Such a colloidosome is depicted in FIG. 11 (multilayer shell: ‘first fluid’ is water, ‘first solvent’ is dodecane/ethanol). The interface may be removed if desired to a permeable, multilayered colloidosome.

### EXAMPLE 7

### Formation of Colloidosomes Utilizing Functionalized Polystyrene Colloidal Particles

An emulsion of silicon oil droplets was prepared in water by mixing equal volumes of silicon oil and water with 2 g/L SDS. The mixture was pushed through a syringe filter with 1.2-micron diameter holes. The filtration was performed a total of 5 times with the aim of making a fairly uniform distribution of oil droplet sizes (about 5 microns diameter).

A volume of 0.01 ml of the above oil-droplet solution was added to 1 ml of de-ionized water. The SDS concentration at this point was about 0.02 g/L.

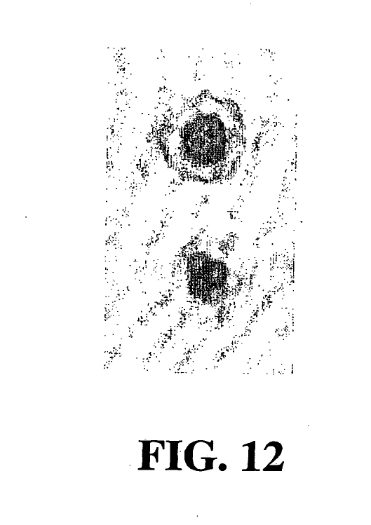

Polystyrene beads (1-micron-diameter and amidine-functionalized) were added to the solution (bead volume fraction about 1%). Within a few minutes, the sample was observed in an optical microscope (see attached figures). Oil droplets that were about 5 microns in diameter were observed. some of the oil droplets were fully coated with amidine beads. Not all droplets were fully coated, however, possibly due to an insufficient quantity of SDS on the droplets. Brighffield optical micrographs were taken of the resulting colloidosomes that were formed and are seen in FIG. 12.

Although not intending to be bound by any particular theory, it is believed that the amidine beads were drawn to and/or held at the oil-water interface by electrostatic attraction. The beads are cationic and the interface is anionic due to the SDS.

In a first control experiment with no SDS in the water, no particles were observed at the oil-water interface. This may be due to the fact that there was nothing to attract the beads to the interface.

In a second control experiment, excess SDS (2 g/L) was used. Droplets were observed, but there were no particles at the oil-water interface. It is possible that the particles became completely coated by SDS, became anionic and were no longer attracted to the interface.

### EXAMPLE 8

### Encapsulation of Active Agents in Colloidosomes

This example shows how a rat fibroblast may be encapsulated in a colloidosome as described herein. The procedure is identical to example 2, except that the aqueous phase contain rat 3T3 fibroblasts (supplied by Justin Jiang, Harvard University). Cells were cultured in Hank's buffered saline (cat #14025-092, Life Technologies, Rockville, Md.). About 0.1 mL of cell/buffer solution was injected into the decalin solution which contained PMMA beads, 0.7 micron diameter, about 1-2 volume %.

Colloidosome solution and control (cell and buffer in Petri dish) were stored in an incubator with controlled temperature and atmosphere. “Control” cells and droplet-encapsulated cells were compared visually using an optical microscope after 30 minutes and 90 minutes. In both cases, cells appeared round in shape, a general indicator of good health (dying cells are distinguishable by shape).

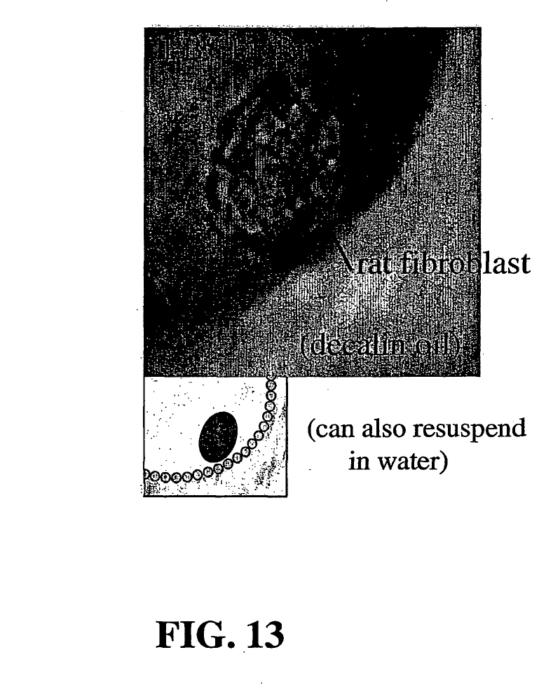

The cells were seen to adhere and spread on the surface of the petri dish (an indicator of good health) and were seen to adhere on the inner (aqueous) surface of the coated droplets, as seen in FIG. 13.

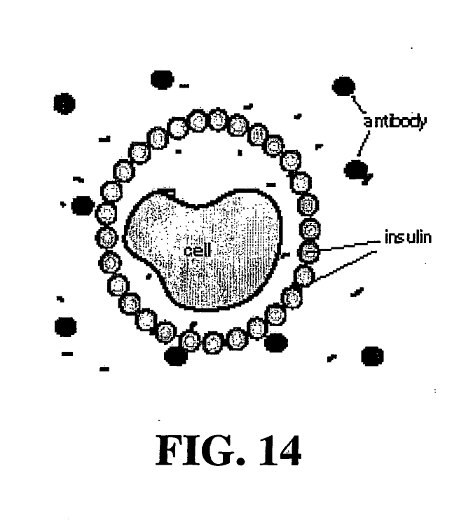

FIG. 14 is a drawing that depicts a cross-section of a colloidosome with an encapsulated pancreatic cell that secretes insulin. As seen in the figure, antibodies are prevented from entering the colloidosome whereas insulin can exit the colloidosome.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method of forming colloidosomes, comprising:
(a) providing particles formed from a biocompatible material in a first solvent;
(b) forming an emulsion by adding a first fluid to said first solvent, said emulsion defined by droplets of said first fluid surrounded by said first solvent;
(c) coating the surface of said droplets with said particles; and
(d) stabilizing said particles on said surface of said droplet to form colloidosomes having a yield strength of at least about 20 Pascals.

2. The method of claim 1, wherein said first solvent is an aqueous solvent and said first fluid is an organic solvent.

3. The method of claim 1, wherein said first solvent is an organic solvent and said first fluid is an aqueous solvent.

4. The method of claim 1, wherein said first solvent is an organic solvent or an aqueous solvent and said first fluid is a gas.

5. The method of claim 1, wherein said particles are substantially spherical.

6. The method of claim 1, wherein said biocompatible material is a polymer.

7. The method of claim 6, wherein said polymer is hydrophobic.

8. The method of claim 7, wherein said hydrophobic polymer is polymethylmethacrylate.

9. The method of claim 7, wherein said hydrophobic polymer is polystyrene.

10. The method of claim 7, wherein said hydrophobic polymer is selected from the group consisting of polystyrene, polymethylmethacrylate, polyalkylenes, silica and combinations thereof.

11. The method of claim 7, wherein said polymer is functionalized with an ionic functional group.

12. The method of claim 11, wherein said functional group is anionic and is selected from the group consisting of carboxyl and sulfate.

13. The method of claim 1, further comprising transferring said colloidosomes into a second fluid and recovering intact colloidosomes, wherein said second fluid is substantially identical to said first fluid.

14. The method of claim 1, wherein at least about 99% of said colloidosomes remain intact after transferring said colloidosomes from said first solvent into a second solvent substantially the same as said first fluid.

15. The method of claim 1, wherein said stabilizing is performed by adding a polyelectrolyte to said first fluid.

16. The method of claim 15, wherein said polyelectrolyte is a polycationic agent.

17. The method of claim 16, wherein said polycationic agent is polylysine.

18. The method of claim 16, wherein said polyelectrolyte is added to said first fluid prior to formation of said emulsion.

19. The method of claim 1, wherein said stabilizing is performed by swelling said particles by adding a second solvent to said first solvent.

20. The method of claim 16, wherein said second solvent is a combination of at least two solvents.

21. The method of claim 1, wherein said stabilizing is performed by a sintering process.

22. The method of claim 1, further comprising isolating said colloidosomes by centrifuging said colloidosomes from said first solvent into a second solvent substantially the same as said first fluid.

23. A method of forming a colloidosome, comprising:
(a) providing substantially spherical particles formed from a biocompatible polymer in a first solvent;
(b) forming an emulsion by adding a first fluid to said first solvent, said emulsion defined by droplets of said first fluid surrounded by said first solvent;
(c) coating the surface of said droplets with said spherical particles; and
(d) stabilizing said particles on said surface of said droplets to form colloidosomes having a yield strength of at least about 20 Pascals.

24. The method of claim 23, wherein said stabilizing is performed by a sintering process.

25. The method of claim 23, wherein said stabilizing is performed by addition of an agent to said first solvent selected from the group consisting of a polycationic agent, a chemical crosslinking agent and a combination thereof.

26. The method of claim 23, wherein said first solvent is an organic solvent and said first fluid is an aqueous solvent.

27. The method of claim 23, wherein said first solvent is an aqueous solvent and said first fluid is an organic solvent.

28. The method of claim 23, wherein said first solvent is an aqueous solvent or an organic solvent and said first fluid is a gas.

29. The method of claim 23, wherein said biocompatible polymer is a hydrophilic polymer.

30. The method of claim 23, wherein said biocompatible polymer is a hydrophobic polymer.

31. The method of claim 30, wherein said hydrophobic polymer is selected from the group consisting of polystyrene, polymethylmethacrylate, polyalkylenes, silica and combinations thereof.

32. A method of encapsulating an active agent, comprising:
(a) providing particles formed from a biocompatible material in a first solvent;
(b) forming an emulsion by adding a second solvent containing said active agent to said first solvent, said emulsion defined by droplets of said second solvent surrounded by said first solvent;
(c) coating the surface of said droplets with said particles; and
(d) stabilizing said particles on said surface of said droplet to form colloidosomes having a yield strength of at least about 20 Pascals.

33. The method of claim 32, wherein said first solvent is an aqueous solvent and said second solvent is an organic solvent.

34. The method of claim 32, wherein said first solvent is an organic solvent and said second solvent is an aqueous solvent.

35. The method of claim 32, wherein said particles are substantially spherical.

36. The method of claim 32, wherein said biocompatible material is a polymer.

37. The method of claim 32, wherein said polymer is hydrophobic.

38. The method of claim 37, wherein said hydrophobic polymer is polymethylmethacrylate.

39. The method of claim 37, wherein said hydrophobic polymer is polystyrene.

40. The method of claim 37, wherein said hydrophobic polymer is selected from the group consisting of polystyrene, polymethylmethacrylate, polyalkylenes, silica and combinations thereof

41. The method of claim 37, wherein said polymer is functionalized with an ionic functional group.

42. The method of claim 41, wherein said functional group is anionic and is selected from the group consisting of carboxyl and sulfate.

43. The method of claim 32, wherein at least about 95% of said colloidosomes remain intact after transferring said colloidosomes from said first solvent into a third solvent substantially the same as said second solvent.

44. The method of claim 32, wherein at least about 99% of said colloidosomes remain intact after transferring said colloidosomes from said first solvent into a third solvent substantially the same as said second solvent.

45. The method of claim 32, wherein said stabilizing is performed by adding a polyelectrolyte to said first solvent.

46. The method of claim 45, wherein said polyelectrolyte is a polycationic agent.

47. The method of claim 46, wherein said polycationic agent is polylysine.

48. The method of claim 32, wherein said stabilizing is performed by swelling said particles by adding a fourth solvent to said first solvent.

49. The method of claim 48, wherein said fourth solvent is a combination of at least two solvents.

50. The method of claim 32, wherein said stabilizing is performed by a sintering process.

51. The method of claim 32, wherein said active agent is selected from the group consisting of a chemical agent or biological agent.

52. The method of claim 51, wherein said chemical agent is selected from the group consisting of a drug, a flavoring agent, a fragrance-producing chemical and a combination thereof.

53. The method of claim 51, wherein said biological agent is a biological macromolecule.

54. The method of claim 51, wherein said macromolecule is selected from the group consisting of a protein, a nucleic acid, a carbohydrate, a lipid and a combination thereof.

55. The method of claim 52, wherein said biological agent is a biological cell.

56. A method of encapsulating an active agent, comprising:
(a) providing substantially spherical particles formed from a biocompatible polymer in a first solvent;
(b) forming an emulsion by adding a second solvent containing said active agent to said first solvent, said emulsion defined by droplets of said second solvent surrounded by said first solvent;
(c) coating the surface of said droplets with said spherical particles; and
(d) stabilizing said particles on said surface of said droplet to form a colloidosome.

57. The method of claim 56, wherein said biocompatible material is selected from the group consisting of polystyrene, polymethylmethacrylate, polyalkylenes, silica and combinations thereof.

58. The method of claim 56, wherein said active agent is a chemical agent or biological agent.

59. The method of claim 58, wherein said chemical agent is selected from the group consisting of a drug, a flavoring agent, a fragrance-producing chemical and a combination thereof.

60. The method of claim 58, wherein said biological agent is a biological macromolecule.

61. The method of claim 60, wherein said macromolecule is selected from the group consisting of a protein, a nucleic acid, a carbohydrate, a lipid and a combination thereof.

62. The method of claim 58, wherein said biological agent is a biological cell.

63. A colloidosome, comprising:
a shell formed of biocompatible, substantially spherical particles, each of said particles linked to neighboring particles, said shell defining an inner chamber and having a plurality of pores extending therethrough, said chamber sized for housing an active agent, said colloidosome having a yield strength of at least about 20 Pascals.

64. The colloidosome of claim 63, wherein said colloidosomes have a yield strength of at least about 50 Pascals.

65. The colloidosome of claim 63, wherein said shell is a monolayer.

66. The colloidosome of claim 63, wherein an active agent in a solvent is disposed within said chamber.

67. The colloidosome of claim 66, wherein said active agent is a biological agent or chemical agent.

68. The colloidosome of claim 67, wherein said chemical agent is selected from the group consisting of a drug, a flavoring agent, a fragrance-producing chemical and a combination thereof.

69. The colloidosome of claim 67, wherein said biological agent is a biological macromolecule.

70. The colloidosome of claim 69, wherein said macromolecule is selected from the group consisting of a protein, a nucleic acid, a carbohydrate, a lipid and a combination thereof.

71. The colloidosome of claim 67, wherein said biological agent is a biological cell.

72. The colloidosome of claim 63, wherein each of said particles are linked to neighboring particles by coalescence of regions of the particles.

73. The colloidsome of claim 63, wherein each of said particles is linked to a neighboring particle by chemical cross-linking.

74. A colloidosome suspension, comprising:
a colloidosome suspended in a first solvent, said colloidosome having a shell formed of biocompatible, substantially spherical particles, each of said particles linked to neighboring particles, said shell defining an inner chamber and having a plurality of pores extending therethrough, said chamber sized for housing an active agent and filled with a second solvent that is substantially similar to said first solvent.

75. The colloidosome of claim 74, wherein said colloidosome has a yield strength of at least about 50 Pascals.
